(19) United States
(12) Patent Application Publication
(10) Pub. No.: US 2016/0249793 A1
(43) Pub. Date: Sep. 1, 2016

(54) Capsule Camera Device with Multi-Spectral Light Sources

(71) Applicants: Kang-Huai WANG, Saratoga, CA (US); CAPSO VISION INC., Saratoga, CA (US)

(72) Inventor: Kang-Huai Wang, Saratoga, CA (US)

(21) Appl. No.: 15/033,644

(22) PCT Filed: Dec. 27, 2013
(86) PCT No.: PCT/US13/77899

§ 371 Date: May 1, 2016

(51) Int. Cl. A61B 1/04; A61B 5/07; A61B 1/00; A61B 1/06; A61B 1/31

(52) U.S. Cl. CPC A61B 1/041; A61B 1/0638; A61B 1/0684; A61B 1/31; A61B 1/00011; A61B 5/073

(57) ABSTRACT

A capsule camera device is disclosed, where the capsule camera device comprises multiple light sources with different spectral characteristics for illuminating lumen walls selectively based on a determined capsule location in the gastrointestinal tract. The capsule camera device includes two or more light sources, an image sensor, a battery, a control module and a housing to enclose the above components in a sealed environment. The control module is used to select one or more selected light sources from said at least two light sources depending on a determined capsule location in the GI tract. Only the selected light source or light sources will be used for illumination in the determined capsule location. The light sources may comprise a white light source and a blue light source with a high spectral concentration at about 415 nm or another light source of different spectral characteristics.

### FIELD OF THE INVENTION

The present invention relates to diagnostic imaging inside the human body using a capsule camera device. In particular, the present invention relates to capturing images in the human gastrointestinal track using multi-spectral light sources.

### BACKGROUND AND RELATED ARTS

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Because of the difficulty traversing a convoluted passage, endoscopes cannot reach the majority of the small intestine and special techniques and precautions, that add cost, are required to reach the entirety of the colon. An alternative in vivo image sensor that addresses many of these problems is capsule endoscope. A camera is housed in a swallowable capsule, along with a radio transmitter for transmitting data, primarily comprising images recorded by the digital camera, to a base-station receiver or transceiver and data recorder outside the body. Another autonomous capsule camera system with on-board data storage was disclosed in the U.S. patent application Ser. No. 11/533,304, filed on Sep. 19, 2006.

It is known that different anatomical structure and tissue characteristics may respond to specific light spectrum differently. For example, blood vessels absorb visible light with a principle peak around the 415 nm (i.e., blue light). This makes the blood vessels seen clearly under light with this short wavelength. Accordingly, narrow band imaging has been used for various endoscopic applications such as to detect the hypervascular locations that often characterize the malignancy of precursor of malignancy. It is also known that cancer lesion has higher concentration of certain substances that have different spectrum responses from the nearby tissues. Therefore, relevance between anatomical structure/tissue characteristics and their responses to specific light spectrum can be utilized to detect the existence of possible malignancy or precursor of malignancy.

However, in the capsule endoscope application, when these special light sources are applied in conjunction with its normal light source, it may substantially increase the power consumption due to the additional light sources and corresponding images captured. Usually a white light source is used as the normal light source to illuminate the gastrointestinal tract during image capture. For capsule applications, the capsule camera is powered by small batteries with limited power budget. The batteries are expected to last for hours or even beyond ten hours after the capsule is swallowed. The target areas to be imaged may be the pharynx, esophagus, stomach, small bowel, colon or the entire gastrointestinal tract. The target areas may be a combination of two or more different areas such as the small bowel and colon. The total number of images to be captured may be as many as tens of thousands. The use of multiple spectral light sources may cause a power budget issue and it is desirable to overcome the issue without the need to substantially increase the battery capacity or to lower image quality and/or the number of captured images.

### BRIEF SUMMARY OF THE INVENTION

A capsule camera device is disclosed, where the capsule camera device comprises multiple light sources with different spectral characteristics for illuminating lumen walls selectively based on a determined capsule location in the gastrointestinal (GI) tract. The capsule camera device includes two or more light sources, an image sensor, a battery, a control module and a housing to enclose the above components in a sealed environment. The control module is used to select one or more selected light sources from said at least two light sources depending on a determined capsule location in the GI tract. Only the selected light source or light sources will be used for illumination in the determined capsule location.

One aspect of the present invention addresses the multiple light sources with different spectral characteristics. The light sources may comprise a white light source and a blue light source, where the white light source has a light spectrum that substantially covers visible lights and the blue light source has a high spectral concentration at about 415 nm. The light sources may comprise an additional light source having high spectral concentration substantially different from 415 nm. The light sources may also comprise two light sources, where one has a high spectral concentration at about 415 nm and the other has a high spectral concentration substantially different from 415 nm. The selected light sources may be used one at a time in turn for illumination in the determined capsule location. The selected light sources may also be used at the same time for illumination in the determined capsule location.

Another aspect of the present invention addresses the determining means to determine the determined capsule location. In one embodiment, the determined capsule location is determined based on expected transit times of the capsule camera using a timing device inside the capsule camera device. In another embodiment, a processing module is used to determine the determined capsule location based on the images captured by the capsule camera device. The processing module can be configured to determine the determined capsule location based on multiple frames of the images captured by the capsule camera device. A determining process for determining the determined capsule location may use a training procedure by correlating said determining the determined capsule location with visual location determination based on the images captured by the capsule camera device. The processing module may be configured to determine the determined capsule location according to color characteristics of the images captured by the capsule camera device. The processing module may be configured to determine the determined capsule location by comparing emitted light intensities by the selected light sources and pixel intensities of the images captured by the capsule camera device. The processing module may be inside the capsule camera device. The processing module may also be in an external device, and the capsule camera device and the external device communicate wirelessly. The determined capsule location may be determined by disposing a location marking device at an intended location on the torso of a patient near the capsule location to be determined in the GI tract. A magnet can be used as the location marking device. The determined capsule location in the GI tract may correspond to the pharynx, esophagus, stomach, small bowel or colon.

In one application, the capsule camera device comprises on-board memory to store the images captured by the capsule camera device. In another application, the capsule camera device comprises a wireless transmitter to transmit the images captured by the capsule camera device to an external receiver.

### BRIEF DESCRIPTION OF THE DRAWINGS

FIG. 1A illustrates exemplary multi-spectral light source arrangement, where white light sources and blue light sources are arranged in a ring around the optics in a forward-looking capsule camera device.

FIG. 1B illustrates exemplary multi-spectral light source arrangement, where white light sources and two types of special light sources with different spectral characteristics are arranged in a ring around the optics in a forward-looking capsule camera device.

FIG. 1C illustrates exemplary multi-spectral light source arrangement, where two types of special light sources with different spectral characteristics are arranged in a ring around the optics in a forward-looking capsule camera device.

FIG. 2A illustrates an exemplary light module consisting of two light sources with different spectral characteristics.

FIG. 2B illustrates exemplary multi-spectral light source arrangement, where multiple light modules consisting of two light sources with different spectral characteristics are used.

FIG. 3 illustrates exemplary multi-spectral light source arrangement along with a control module, where a timing module is used to determine the capsule location based on expected transit time.

FIG. 4 illustrates exemplary multi-spectral light source arrangement along with a control module, where an image-based location module inside the capsule camera device is used to determine the capsule location.

FIG. 5 illustrates exemplary multi-spectral light source arrangement along with a control module, where an image-based location module in an external device is used to determine the capsule location, and the external device and the capsule camera device communicate wirelessly.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely representative of selected embodiments of the invention.

Reference throughout this specification to “one embodiment,” “an embodiment,” or similar language means that a particular feature, structure, or characteristic described in connection with the embodiment may he included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention.

The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

The use of he special spectrum light source or a second light source is targeted for a certain type of lesion, cancerous, pre-cancerous, or otherwise abnormal condition. Therefore, the use of the special spectrum light source or a second light source should be limited to the intended sections of the gastrointestinal (GI) tract. For example, a patient has certain symptoms indicative of a certain section with pathologies or a patient has familial inheritance of a certain disease that normally exists in a certain section of the GI tract. In another example, a patient has some particular habits such as smoke or volume alcohol consumption that may result in certain section in the GI tract in need of special attention. In yet another example, a patient may have a certain pre-exiting condition such as GERD (Gastroesophageal reflux disease), helicobacter pylori or previous colon polyp removal, which makes this patient a higher risk of malignancy in the esophagus, stomach or colon respectively. In yet another example, a certain section of the GI tract has higher dangerous pathologies than the other sections, such as the colon has higher likelihood of cancer growth than the small bowel.

Furthermore, the lesion in different sections of the GI tract may have different spectral response characteristics. Therefore, different light sources or different groups of light sources may be needed for each section. According to the present invention, different light sources having different spectral contents are applied to different sections of the GI tract. This may result in images more clearly indicative of pathologic significance and can also achieve substantial power saving compared to the case of using all light sources indiscriminately.

White light is usually used for standard endoscopy, where the light source has a light spectrum that covers substantially all visible lights. Therefore, white light endoscope can produce images similar to visual inspection of the mucosa with the naked eye. The LED light is typically used for the capsule application due to its compactness and low power consumption.

In one embodiment, images for an intended area of the GI tract may be taken under more than one light source, where each light source has a certain spectral characteristics. For example, a certain cancer lesion has different spectral responses. The images with combined light sources may reveal all features that are visible under lights with respective spectral responses. In another example, a section of the GI tract with two types of lesions having different spectral responses may take advantage of light sources with different corresponding spectral characteristics to reveal possible lesion in the images. In this case, light sources with different corresponding spectral characteristics are applied to the section of the GI tract where the two types of lesion are prone to occur. In yet another example, different images may be taken with one or more white light sources, one or more first special light sources with first spectral characteristic, and one or more second special light sources with second spectral characteristic for that section. The white light sources, the first special light sources and the second special sources are applied in turn to illuminate the lumen during image capture. For example, the white light sources may be applied to capture the first images the first special light sources may be used next to capture the second image; and then the second special light sources may be used to capture the third image for a substantially same scene. While the different types of light sources may be used in turn, these light sources may also be applied in a combined fashion. For example, the white light sources and the first special light sources may be used at the same time to illuminate a particular section of the GI tract. The white light sources and the second special light sources may be used at the same time to illuminate another particular section of the GI tract.

The capsule travels through the entire GI tract in a certain definitive order from the mouth into the pharynx, through the esophagus, then into the stomach, through the small intestine and finally through colon, before excretion. There is a wide variation of transit time in each section within population. For example, the length of time for the capsule to stay in the stomach before exiting from it may take hours. It may take 2 to 10 hours to travel through the small intestine depending on the motility of each individual. Even for a given individual, the transit time may vary significantly from day to day. For vast majority of patients, it usually takes no more than 5 minutes for the capsule to pass through the pharynx and esophagus and 2 hours to exit the stomach, and 12 hours to pass through the small bowel. Accordingly, one embodiment of the present invention utilizes an internal timing scheme to schedule the usage of a corresponding light source, a set of corresponding light sources among multiple light sources, or multiple sets of light sources for a respective target area of the GI tract. Each of the multiple light sources or multiple sets of light sources has a specific light spectral characteristics intended for revealing specific tissue characteristics. The target area may correspond to a GI section such as the pharynx, esophagus, stomach, small intestine or colon. The target area may also correspond to a transitional area such as the pylorus at the bottom end of the stomach toward the small bowel. The multiple light sources or multiple sets of light sources according to the present invention are carefully chosen to match the intended diagnosis. For example, a light source with high spectral concentration at about 415 nm is selected for imaging in the pharynx, esophagus and colon. When a scheduled light source selection is used, the light source with high 415 nm concentration can be selected for imaging from 1 to 5 minutes and from 2 hours after the capsule is swallowed. If the stomach is the target area to be imaged, a corresponding light source or a set of light sources can he selected in the first hour to examine stomach effectively.

In order to adapt to the transit time variation, another embodiment of the present invention utilizes image-based location determination to determine the arrival of capsule in a designated section in the GI tract. The images captured by the capsule camera may be indicative of the capsule location. For example, the size of the lumen can be judged by comparing the light intensity emitted from the light sources and the pixel intensities of the captured images. When the ratio of light intensity to the received image pixel intensity decreases noticeably, the capsule has presumably left the more spacious lumen (e.g., the stomach) and gone into a narrow lumen (e.g., the small bowel). For more accurate estimation, the process can be performed over a number of images. Color characteristic may also be used for making such an informed judgment. To deal with inter-person difference in GI color, in one embodiment of using color characteristic to determine the location, the color change from previous images of recent images within the same video of the procedure is used to determine the arrival of a new anatomical section in GI. The image-based location determination can be implemented using software, hardware modules, or a combination of software and hardware. The image-based location determination process can he performed using computational resources within the capsule. In one embodiment, a location determination algorithm is developed or fine-tuned with the help of accurate determination of the capsule location inside the GI tract. For example, a training procedure may be used to adjust the location determination algorithm by correlating the results from the algorithm and the result from an accurate location determination mechanism. For example, a human expert may visually examine the captured image and make more accurate location determination. The location determination algorithm may be realized in software program or programmable processor. For a capsule equipped with a wireless communication link between the capsule and a receiving station external to the human body, the required computation to determine the arrival of the capsule at a designated section of the GI tract can be performed externally. The capsule can be configured accordingly to use a desired light source or a set of light sources by the transmission from an external device of a determined location or a control signal back to the capsule.

In yet another embodiment, one or more location marking device can be placed external to human body at the proper location or locations. The capsule can be configured to detect the arrival at the pre-determined location or locations. Upon the detection of the pre-determined location or locations, the capsule can select a particular light source or a particular group of light sources to illuminate the lumen. For example, a magnet can be used as the location marking device and placed on the abdomen corresponding to the transition between the small bowel and the colon. When the capsule comes to the proximity of the magnet, the capsule may sense the existence of the magnet and select a desirable light source or a desirable set of light sources. Alternatively, an external device for location determining can be used to detect the capsule arrival in a certain location. Upon the detection of capsule arrival at the designated location, the external device can transmit a signal to the capsule wirelessly to cause the capsule to select a desirable light source or a desirable set of light sources.

Narrow band imaging (NBI) has been found useful to improve detection of cancerous tissue. For example, in a study by Emura, et al., (“The pharynx: examination of an area too often ignored during upper endoscopy”, published in GASTROINTESTINAL ENDOSCOPY, 2013, Vol. 78, No. 1, pages 143-149), NBI has been shown to be able to facilitate the detection of pharyngeal cancers. The use of NBI has remarkably improved visualization of superficial vessels over the conventional white-light endoscopy, allowing easier identification of hypervascular sites and early cancers. The NBI technique is also applicable to improve detection of cancer in other parts of the GI tract such as the esophagus, stomach, small bowel and colon.

In the field of endoscopy, there is a technique for discriminating between normal and adenomatous tissues based on autofluorescence excitation-emission matrix (EEM). In autofluorescence EEM, the emission spectrum at one or more certain excitation wavelengths may be used to illuminate the underlying tissues. The captured images can be examined in reference to the spectra of the excitation light to determine possible adenomatous. For example, in a study by Li, et al., (“Autofluorescence excitation-emission matrices for diagnosis of colonic cancer”, published in World Journal of Gastroenterology, July 2005, Vol. 11, No. 25, pages 3931-3934), the spectral response of images with any excitation wavelength higher than 380 nm illustrates very distinct characteristics between a normal tissue and a cancerous tissue. Accordingly, one or more corresponding light sources (e.g., one with 460 nm and one with 540 nm wavelengths) may be used to illuminate the colon. The autofluorescence EEM technique can also be applied to detect anomaly in other parts of the GI tract such as the pharynx, esophagus or small bowel with a respective light source or a set of light sources. Under a similar principle to the autofluorescence EEM, light sources with high spectral concentration at selected wavelengths can be used to illuminate a particular section of the GI track. The autofluorescence EEM can be obtained from the captured images and the known spectral characteristics of the light sources. Consequently, the present invention is also applicable to the autofluorescence EEM application.

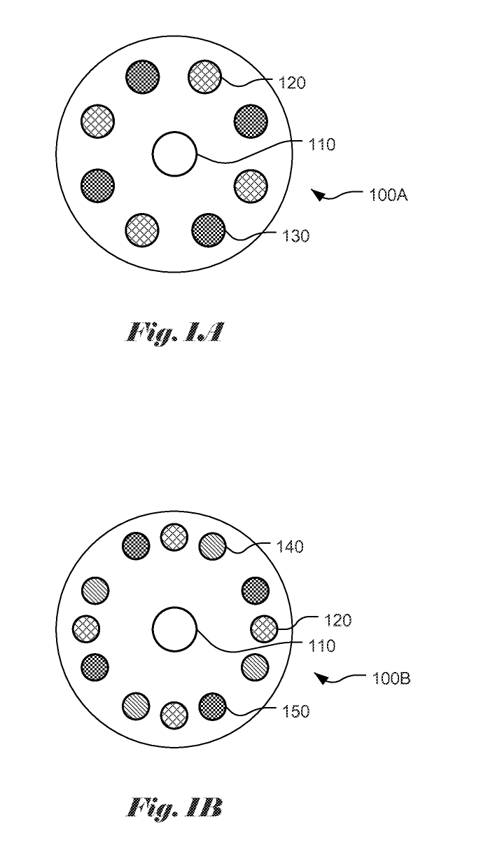

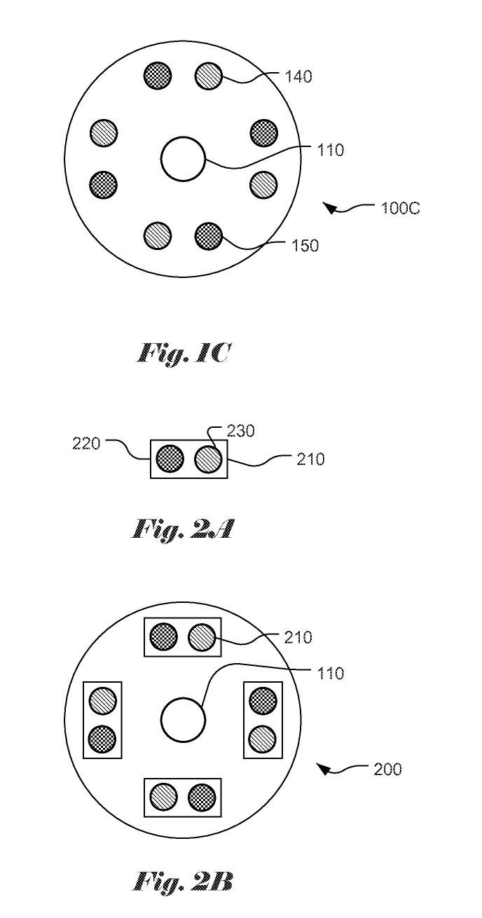

According to the present invention, multiple light sources or multiple sets of light sources are used in the capsule camera, where the light sources are selected based on the capsule location in the GI tract. FIG. 1A illustrates one example of a lighting arrangement (100A) for a forward facing capsule camera having normal white light sources (120) and special light sources (130) with high concentration at 415 nm blue light. As shown in FIG. 1A, the light sources arc placed around optics 110 of the capsule camera. Special light sources with high concentration at other wavelengths may also be used. FIG. 1B illustrates another lighting arrangement according to the present invention, where the lighting configuration includes two types of light sources (140 and 150) having high concentrations at two separate wavelengths. Normal light sources (120) are also included in the lighting arrangement. FIG. 1C illustrates yet another lighting arrangement without the normal light sources according to the present invention, where the lighting configuration includes two types of light sources (140 and 150) having two different spectral characteristics. While up to two types of special light sources are used in the above examples, the lighting arrangement according to the present invention may also use more than two types of special light sources. The light sources are arranged in a ring around the optics in FIG. 1. Nevertheless, the light sources may be arranged in other patterns around the optics. For example, the light sources may also be arranged in a matrix pattern around the optics (110).

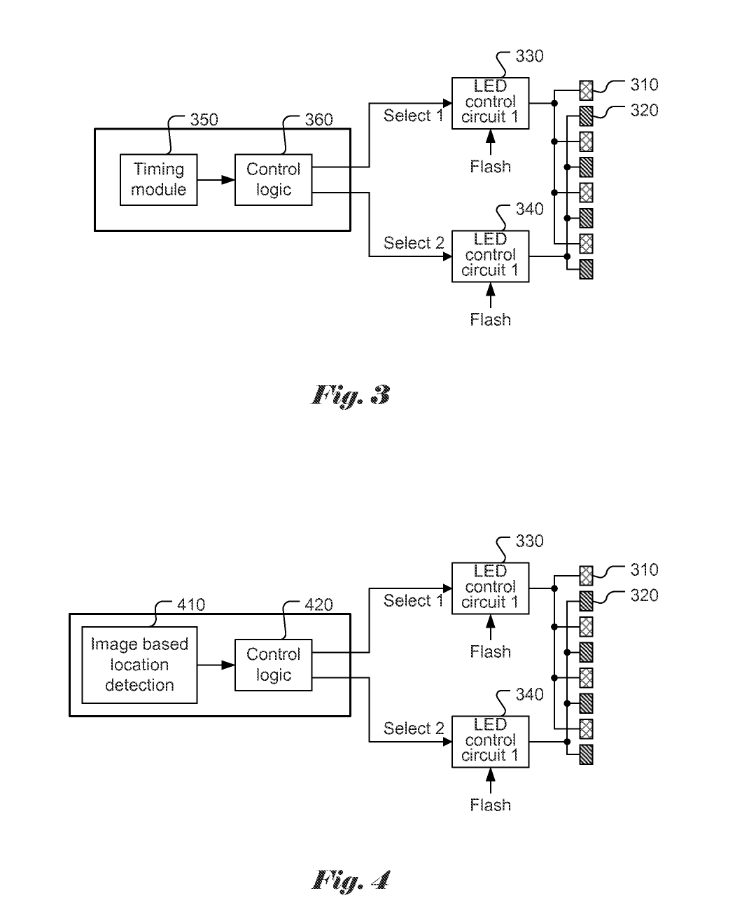

Multiple spectral light sources may be arranged as a multiple spectral light module. A lighting arrangement may use multiple modules. For example, FIG. 2A illustrates a multiple spectral light module (210) consisting of two light sources (220 and 230) with different spectral characteristics, where the light sources in the light module can be controlled to turn On or Off independently, or driven independently. While two different light sources are used in the example, more than two light sources may also be used. FIG. 2B illustrates an exemplary lighting arrangement (200) incorporating multiple spectral light modules (210) according to an embodiment of the present invention.

FIG. 3 illustrates an exemplary control module that incorporates an embodiment of the present invention to select multiple spectral light sources based on a pre-defined order. A timing device is used for counting the elapse time after the capsule is swallowed. Two different types of light sources (310 and 320) are used in this example, where each type of light sources consists of 4 LED lights and each set has its corresponding LED control/driver circuit (330 or 340). Control signals (Select 1 and Select 2) are used to select one of the two types of light sources. As mentioned before, a timing device such as a timer can be used to trigger control signals based on anticipated capsule transit times in the GI tract. Upon the triggered signals from the timer (350) associated with the expected capsule locations in the GI tract, the control logic (360) will generate control signals to select a corresponding type of light sources. For example, lights 310 correspond to normal lights and lights 320 correspond to blue lights with spectrum peak at 415 nm. The control circuit may select both normal light (310) and blue lights (320) when the capsule is expected to be in the pharynx, esophagus or colon. In this case, the two types of light sources can be used in an interleaving fashion, i.e., imaging the target GI tract using one of the two types of lights followed by the other of the two types of lights and repeating the pattern. When the capsule is expected to be in the small bowel and the small bowel is not the target area, the control logic may select only the normal lights in this case. Since the light sources will be used to illuminate the GI tract only when images are taken, the “Flash” signal is used to control when to trigger the light sources.

In the example of FIG. 3, the multiple spectral light sources and associated control sub-system is configured as a control modules with a tinier and control logic, and two sets of light sources with respective LED control circuit. However, the sub-system can be partitioned differently to practice the present invention without departing from the spirit of the present invention. For example, LED control circuit 1 and LED control circuit 2 can be implemented along with the timer and control logic as part of the control module. Alternatively, LED control circuit 1 and LED control circuit 2 may also be packaged with the respective light sources. Furthermore, the control module may also be implemented based on common processing resources in the capsule device. For example, control logic 360 may correspond to a functional module implemented by a controller or a processor in the capsule device, where the controller or the processor also performs other tasks such as image capture control.

In another embodiment, each LED could be driven individually for different light intensity and/or duration of On time, where the abovementioned Off state is synonymous to driving strength is substantially zero or the driving duration is substantially zero.

FIG. 4 illustrates an exemplary control module that incorporates an embodiment of the present invention to select multiple spectral light sources based on on-board image-based location detection. As mentioned earlier, the images captured inside the GI tract can be used to determine the section of the GI tract that the capsule is located. There are several know techniques in the field to determine a respective GI section based on the captured images. For example, the spectral characteristics of the capsule images can he used to determine the respective GI section (e.g., stomach or small bowel). The reflected light intensity from a known emitted light intensity can also be used to discriminate whether the capsule is in the stomach, small bowel or colon. As shown in FIG. 4, image-based location detection (410) is used to determine the section of the GI that the capsule is located. Images or a portion of images are provided to the image-based location detection module. The detection result is then used by the control logic (420) to select the respective light sources.

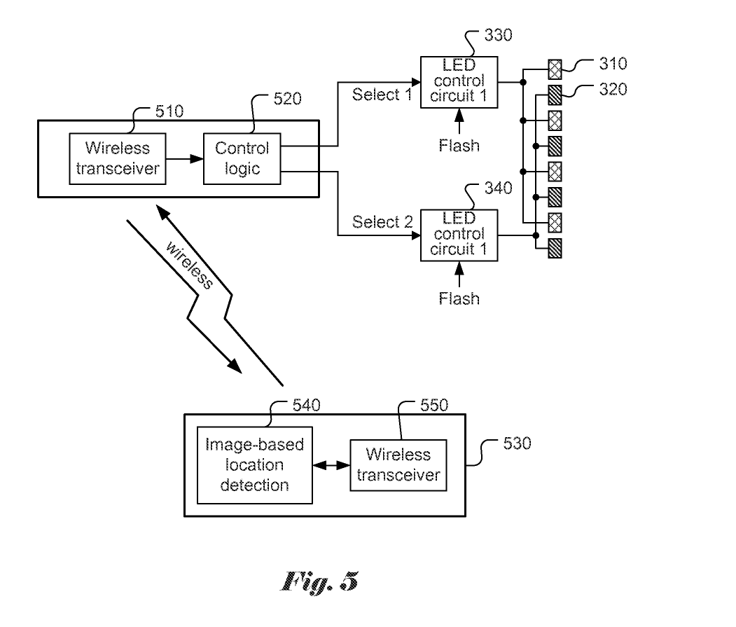

While FIG. 4 illustrates an example of image-based location detection using an on-board processing module, image-based location detection may also be performed by an external device for a capsule device equipped with a wireless transceiver to transmit captured images to the external device. FIG. 5 illustrates an exemplary system according to an embodiment of the present invention. The capsule device includes a transceiver (510) to transmit captured images to an external device 530. The external device comprises a wireless transceiver (550) and an image-based location detection module (540). The images are received by the wireless transceiver (550) of the external device. The received images are then processed by the image-based location detection module (540). The result is transmitted from the wireless transceiver (550) of the external device back to the capsule. The wireless transceiver (510) in the capsule device receives the location information or control information from the external device and the location information or control is passed to the control logic (520).

The invention may he embodied in other specific forms without departing from its spirit or essential characteristics. The described examples are to be considered in all respects only as illustrative and not restrictive. The scope of the invention is therefore indicated by the appended claims rather than by the foregoing description. All changes which come within the meaning and range of equivalency of the claims are to be embraced within their scope.

1. A capsule camera device, comprising:
at least two light sources to illuminate lumen walls of a GI (gastrointestinal) tract, wherein said at least two light sources have different spectral characteristics;
an image sensor to capture images from a scene illuminated by said at least two light sources;
a battery to provide power to the capsule camera device;
a control module to select one or more selected light sources from said at least two light sources depending on a determined capsule location in the GI tract, wherein only said one or more selected light sources are used for illumination in the determined capsule location; and
a housing to enclose said at least two light sources, the image sensor, the battery and the control module in a sealed environment.

2. The capsule camera device of claim 1, wherein said at least two light sources comprise a first light source corresponding to a white light source and a second light source corresponding to a blue light source, wherein the white light source has a light spectrum that substantially covers visible lights and the blue light source has a high spectral concentration at about 415 nm.

3. The capsule camera device of claim 2, wherein said at least two light sources comprise a third light source, wherein the spectral characteristics of the third light is substantially different from the spectral characteristics of the first source and the second light source.

4. The capsule camera device of claim 1, wherein said at least two light sources comprise a first light source and a second light source, wherein the first light source has a first high spectral concentration at about 415 nm and the spectral characteristics of the second light source is substantially different from the spectral characteristics of the first light source.

5. The capsule camera device of claim 1, wherein said one or more selected light sources are used one at a time sequentially for illumination in the determined capsule location when a number of said one or more selected light sources is more than one.

6. The capsule camera device of claim 1, wherein said one or more selected light sources are used at a same time for illumination in the determined capsule location when a number of said one or more selected light sources is more than one.

7. The capsule camera device of claim 1, wherein the determined capsule location is determined based on expected transit times of the capsule camera using a liming device in the capsule camera device.

8. The capsule camera device of claim 1, wherein a processing module is configured to determine the determined capsule location based on the images captured by the capsule camera device.

9. The capsule camera device of claim 8, wherein the processing module is configured to determine the determined capsule location based on multiple frames of the images captured by the capsule camera device.

10. The capsule camera device of claim 8, wherein the processing module includes a location determining means, wherein the location determining means utilizes previously derived information correlating location results determined by initial location determining means and by visual determination.

11. The capsule camera device of claim 8, wherein the processing module is configured to determine the determined capsule location according to color characteristics of the images captured by the capsule camera device.

12. The capsule camera device of claim 8, wherein the processing module is configured to determine the determined capsule location by comparing emitted light intensities by said one or more selected light sources and pixel intensities of the images captured by the capsule camera device.

13. The capsule camera device of claim 8, wherein the processing module is inside the capsule camera device.

14. The capsule camera device of claim 8, wherein the processing module is in an external device, wherein the capsule camera device and the external device communicate wirelessly.

15. The capsule camera device of claim 1, wherein the determined capsule location in the GI tract corresponds to a GI part selected from a group consisting of pharynx, esophagus, stomach, small bowel and colon.

16. The capsule camera device of claim 1, wherein the determined capsule location is determined by disposing a location marking device at an intended location on torso of a patient near the determined capsule location in the GI tract.

17. The capsule camera device of claim 16, wherein the location marking device corresponds to a magnet.

18. The capsule camera device of claim 1, wherein the determined capsule location is determined by an external device disposed at an intended location on torso of a patient near the determined capsule location in the GI tract, wherein the external device detects the capsule device in a proximity of the capsule device and communicates proximity information to the capsule device.

19. The capsule camera device of claim 1, further comprising on-board memory to store the images captured by the capsule camera device.

20. The capsule camera device of claim 1, further comprising a wireless transmitter to transmit the images captured by the capsule camera device to an external receiver.
